(19) United States
(12) Patent Application Publication
(10) Pub. No.: US 2018/0120285 A1
(43) Pub. Date: May 3, 2018

(54) Hand Tool for Identifying Dry Rot Damage in Wood

(71) Applicant: Brian Wickstrom, Woodland, CA (US)

(72) Inventor: Brian Wickstrom, Woodland, CA (US)

(21) Appl. No.: 15/590,126

(22) Filed: May 9, 2017

Related U.S. Application Data
(60) Provisional application No. 62/414,693, filed on Oct. 29, 2016.

(51) Int. Cl. G01N 33/46; B25G 1/10

(52) U.S. Cl. CPC G01N 33/46; B25G 1/102; G01N 2203/0076; G01N 2203/0019; G01N 2203/0041; B25G 1/10

(57) ABSTRACT

A hand tool for use in identifying dry rot damage in wood during an inspection includes an elongated rod having a first end portion and a second end portion, the first end portion having a tapered portion extending to a blunt end, a handle disposed around the rod and extending from an intermediate portion of the rod to the second end of the rod, and a grip overmold layer disposed around the handle. The tool is maneuvered to permit the blunt end of the rod to contact the wood to identify the presence of dry rot damage therein during the inspection.

### REFERENCE TO RELATED APPLICATIONS

This application claims the benefit of provisional patent application 62/414,693 filed Oct. 29, 2016 titled Hand Tool For Identifying Dry Rot Damage In Wood, which is incorporated herein in its entirety by reference.

### BACKGROUND OF THE INVENTION

This invention relates generally to tools for identifying dry rot damage in wood.

Wood with dry rot fungus damage can be difficult to locate visually. Many times it is hidden between two pieces of wood or beneath multiple layers of paint. Building inspectors typically examine the wood siding of buildings for dry rot damage by using various tools such as sticks, paint roller cages, and other devices with poles that may extend 6 to 8 feet long. The end of each device is pushed against the wood. If the wood caves in and pushes through, the presence of dry rot damage is likely. On the other hand, wood that does not cave is indicative that the wood is in an acceptable condition.

Although presumably effective for their intended purposes, the existing devices have several limitations in that they are difficult to grip and/or difficult to maneuver during the inspection process. Therefore, it would be desirable to have a hand tool for identifying dry rot damage in wood that is easy to grip, has a threaded end configured to receive an extension member, and having a blunt end that only causes damage if the wood it is pressed against crumbles due to dry rot.

### SUMMARY OF THE INVENTION

Accordingly, a hand tool for use in identifying dry rot damage in wood during an inspection according to the present invention includes an elongated rod having a first end portion and a second end portion, the first end portion defining a tapered portion extending to a blunt end. A handle is disposed around the rod and extends from an intermediate portion of the rod to the second end of the rod, and a grip overmold layer is disposed around the handle. In use, the tool is maneuvered to permit the blunt end of the rod to contact the wood to identify the presence of dry rot damage therein during the inspection.

Therefore, a general object of this invention is to provide a hand tool for use in identifying dry rot damage in wood.

Another object of this invention is to provide a hand tool, as aforesaid, that includes an elongated linear rod having a blunt end that will either penetrate into rotten wood when pressed against a wooden surface.

Still another object of this invention is to provide a hand tool, as aforesaid, having an easy to grip overmolded handle.

Yet another object of this invention is to provide a hand tool, as aforesaid, in which the handle defines a threaded area configured to receive an extension device so that the hand tool may be used to inspect remote areas of a structure.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a hand tool for use in identifying dry rot damage in wood according to a preferred embodiment of the present invention;

FIG. 2 is a perspective view from another angle of the hand tool as in FIG. 1;

FIG. 3 is a top view of the hand tool as in FIG. 1;

FIG. 4 is an upper perspective view of the hand tool as in FIG. 1;

FIG. 5 is a side view of the hand tool as in FIG. 3; and

FIG. 6 is a bottom view of the hand tool as in FIG. 3.

### DESCRIPTION OF THE PREFERRED EMBODIMENT

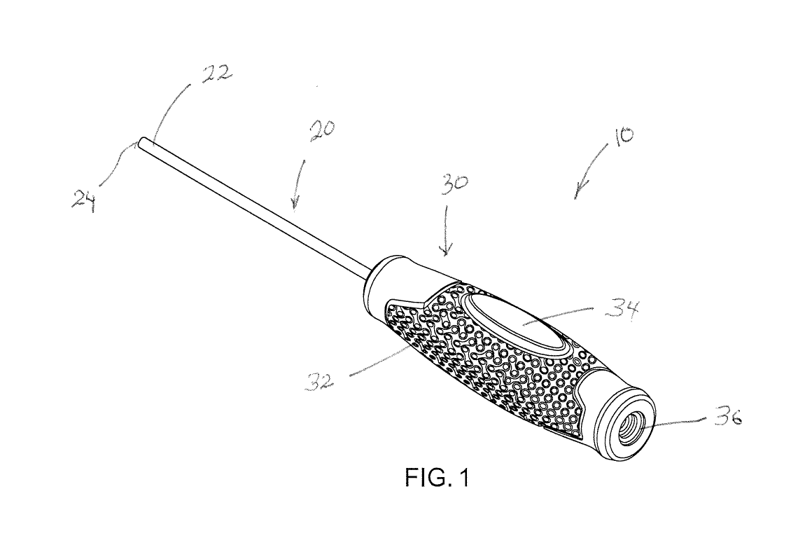

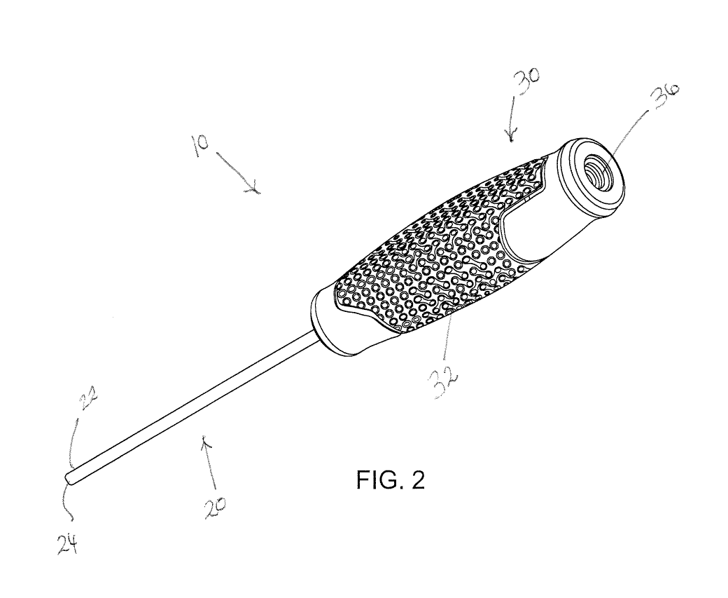

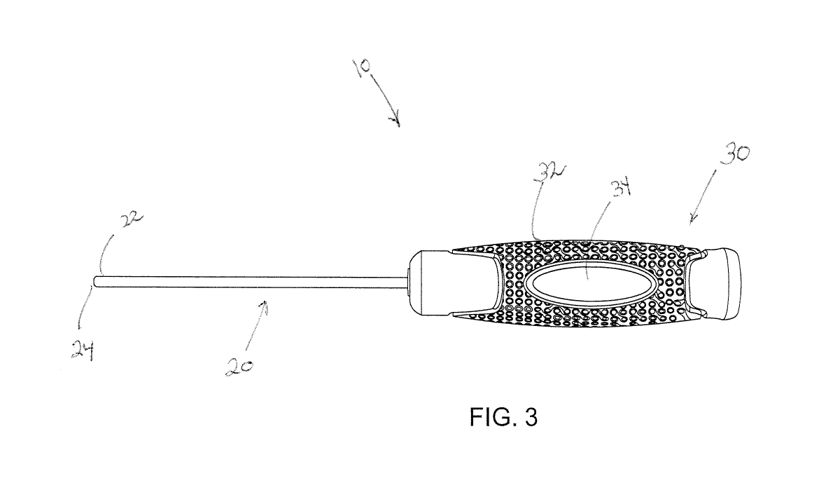

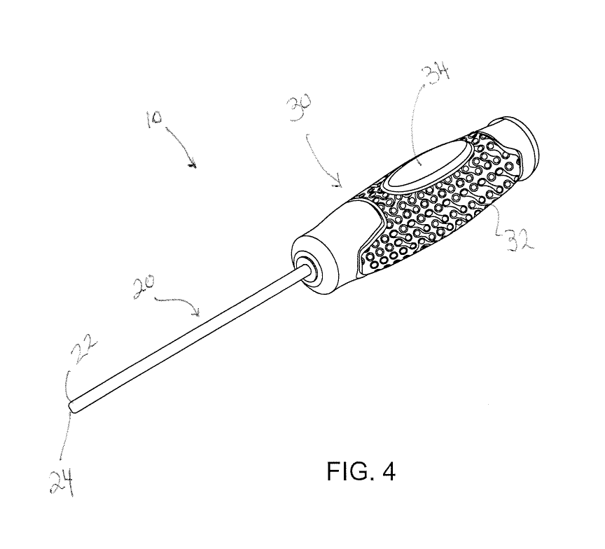

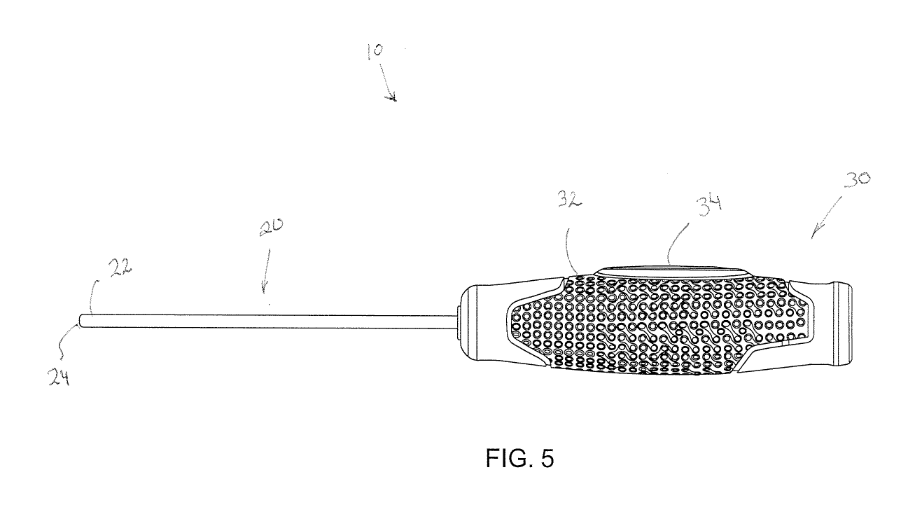

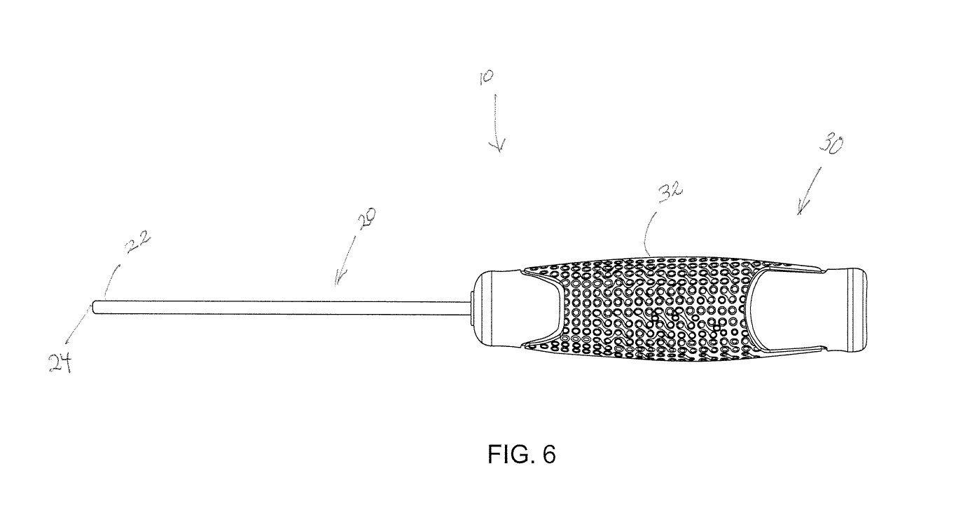

A hand tool for use in identifying dry rot damage in wood according to a preferred embodiment of the present invention will now be described in detail with reference to FIGS. 1 to 6 of the accompanying drawings.

The hand tool 10 includes a rod 20 having a generally linear and elongated configuration. The elongated rod 20 is preferably a chrome plated steel probe. The rod 20 includes a first end portion (not visible as it is inside the handle) and an opposed second end portion 22 having a blunt terminal end 24. A handle 30 is disposed around the rod 20 and extends from an intermediate portion of the rod 20 to the first end portion of the rod 20. The handle 30 may be constructed of glass-filled nylon polymer material or the like.

The handle 30 may include a grip portion 32 that is overmolded upon an exterior surface of the handle 30. Overmolding is a manufacturing or prototyping process of adding one material over another part that has already been made. In an embodiment, the overmolded grip portion 32 may be situated on only a portion of the handle, i.e. not upon the entirety of the exterior of the handle 30. The grip portion 32 may be constructed of a thermoplastic elastomeric material. The grip overmold layer is preferably made from a. textured rubber material such as a thermoplastic elastomer, thus making it a tactile surface that is easy to grip.

In one embodiment, a proximal end of the handle 30 defines a threaded opening 36 having U.S. Standard Female Threads. More particularly, the proximal end of the handle 30 defines an inwardly extending channel in communication with the opening 36, the channel having an inner surface defining the female threads. This threaded opening 36 is configured to receive the male threaded end of an extension pole through the opening 36. This selectively extends the length of the handle 30 to enhance ease of use of the tool. In an embodiment, the channel and opening may be defined by an extension attachment coupled to the proximal end of the handle 30 but is otherwise not integrally constructed with the handle 30.

In another aspect, the grip portion 32 of the handle 30 may include a smooth surface 34 on which indicia may be imprinted. More particularly, indicia such as alpha-numeric indicia, graphical indicia, or even color indicia may be applied to the smooth surface. Exemplary indicia may include advertisements, company logos, athletic team names or mascots, personal identification, and the like.

In operation, the user maneuvers the hand tool 10 to inspect a wood member such as

siding on a building. The user grabs the handle 30 and presses the blunt end against the wood. If the wood caves in and pushes through, the presence of dry rot damage is likely. On the other hand, wood that does not cave in indicates the wood is in an acceptable condition. The extension pole can be secured to the threaded opening 36 in the tool's handle 30. This permits the user to identify dry rot damage in high elevation areas such as roof eaves from the ground level, thus eliminating the need for a ladder.

It shall be appreciated that the hand tool 10 may be useful for wood destroying pest and organism inspectors, home appraisers, house inspectors, and the like. It shall be appreciated that the components of the hand tool described in several embodiments herein may comprise any alternative known materials in the field and be of any color, size and/or dimensions. It shall be appreciated that the components of the hand tool described herein may be manufactured and assembled using any known techniques in the field.

It is understood that while certain forms of this invention have been illustrated and described, it is not limited thereto except insofar as such limitations are included in the following claims and allowable functional equivalents thereof.

1. A hand tool for identifying dry rot damage in wood during an inspection, comprising:
a rod having a linear and elongated configuration and that includes opposed first and second end portions, said first end portion having a blunt end;
a handle disposed around said rod and extending from an intermediate portion of said rod to said second end portion of said rod;
wherein said handle includes a grip portion that is overmolded on an exterior surface of said handle;
wherein said blunt end of said rod is configured to contact the wood so as to identify the presence of dry rot damage therein during the inspection.

2. The hand tool as in claim 1, wherein said grip portion is an overmold layer of textured rubber material positioned on less than an entirety of the exterior surface of said handle.

3. The hand tool as in claim 1, wherein said grip portion is constructed of a thermoplastic elastomer.

4. The hand tool as in claim 1, wherein said handle includes a proximal end defining an opening having female threads configured to receive a threaded male end of an extension member.

5. The hand tool as in claim 4, wherein said proximal end of said handle defines an inwardly extending channel in communication with said opening, said channel having an inner surface defining said female threads configured to receive the threaded male end of the extension member.

6. The hand tool as in claim 1, wherein said handle is constructed of glass-filled nylon polymer material.

7. The hand tool as in claim 6, wherein said rod is chrome-plated.

8. The hand tool as in claim 1, wherein said handle includes a smooth surface disposed in said grip portion, said smooth surface having indicia thereon.

9. The hand tool as in claim 8, wherein said indicia is taken from a group comprising alpha-numeric, graphical, and color indicia.

10. The hand tool as in claim 9, wherein said grip portion of said handle has an ergonomic configuration and constructed of a tactile material.

11. A hand tool for identifying dry rot damage in wood during an inspection, comprising:
a rod having a linear and elongated configuration and that includes opposed first and second end portions, said first end portion having a blunt end; and
a handle disposed around said rod and extending from an intermediate portion of said rod to said second end portion of said rod;
wherein said handle includes a proximal end defining an opening having female threads configured to receive a threaded male end of an extension member;
wherein said handle includes a grip portion constructed of textured rubber material that is overmolded on an exterior surface of said handle;
wherein said blunt end of said rod is configured to contact the wood so as to identify the presence of dry rot damage therein during the inspection.

12. The hand tool as in claim 11, wherein said grip portion is positioned on less than an entirety of the exterior surface of said handle.

13. The hand tool as in claim 11, wherein said grip portion is constructed of a thermoplastic elastomer.

14. The hand tool as in claim 11, wherein said proximal end of said handle defines an inwardly extending channel in communication with said opening, said channel having an inner surface defining said female threads configured to receive the threaded male end of the extension member.

15. The hand tool as in claim 11, wherein said handle is constructed of glass-filled nylon polymer material.

16. The hand tool as in claim 11, wherein said rod is chrome-plated.

17. The hand tool as in claim 11, wherein said handle includes a smooth surface disposed in said grip portion, said smooth surface having indicia thereon.

18. The hand tool as in claim 17, wherein said indicia is taken from a group comprising alpha-numeric, graphical, and color indicia.

19. The hand tool as in claim 9, wherein said handle has an ergonomic configuration and constructed of a tactile material.

20. A hand tool for identifying dry rot damage in wood during an inspection, comprising:
a rod having a linear and elongated configuration and that includes opposed first and second end portions, said first end portion having a blunt end; and
a handle disposed around said rod and extending from an intermediate portion of said rod to said second end of said rod;
wherein:
said handle includes a proximal end defining an opening having female threads configured to receive a threaded male end of an extension member;
said handle includes a grip portion constructed of textured rubber material that is overmolded on an exterior surface of said handle;
said handle includes a smooth surface disposed in said grip portion, said smooth surface having indicia thereon;
said blunt end of said rod is configured to contact the wood so as to identify the presence of dry rot damage therein during the inspection.
